(19) United States
(12) Patent Application Publication
(10) Pub. No.: US 2006/0241515 A1
(43) Pub. Date: Oct. 26, 2006

(54) SINGLE-HAND OPERATED SYRINGE-LIKE DEVICE THAT PROVIDES ELECTRONIC CHAIN OF CUSTODY WHEN SECURING A SAMPLE FOR ANALYSIS

(76) Inventors: Jeffrey L. Jones, Orem, UT (US); Randall W. Waite, Springville, UT (US); Tai Van Truong, Provo, UT (US); Jacolin A. Murray, Farmington, UT (US); Jon A. Kimball, Provo, UT (US); Edgar D. Lee, Highland, UT (US); Milton L. Lee, Pleasant Grove, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 11/379,716

(22) Filed: Apr. 21, 2006

Related U.S. Application Data
(60) Provisional application No. 60/673,745, filed on Apr. 21, 2005.
(60) Provisional application No. 60/673,744, filed on Apr. 21, 2005.

(51) Int. Cl. A61B 10/00; A61B 5/00

(52) U.S. Cl. 600/562; 600/573

(57) ABSTRACT

A syringe-like device (hereinafter “syringe”) that is operable by a single hand, wherein the syringe includes a plunger for ejecting and then retracting a fiber or filament used for the collection of solids, solids in suspensions and liquids, wherein the syringe includes a microchip embedded in the syringe housing, and wherein the microchip enables electronic chain of custody tracking of a sample from a point of origin through final analysis.

### CROSS REFERENCE TO RELATED APPLICATIONS

This document claims priority to and incorporates by reference all of the subject matter included in the provisional patent applications, docket number 05-17, having Ser. No. 60/673,745 and filed on Apr. 21, 2005, and docket number 05-18, having Ser. No. 60/673,744, and filed on Apr. 21, 2005.

### BACKGROUND OF THE INVENTION

1. Field of the Invention

This invention relates generally to a syringe-like device that is used to collect samples for analysis. More specifically, the present invention provides a modified syringe-like device that enables single-handed operation when obtaining samples for analysis, and enables electronic chain of custody of the samples being gathered.

2. Description of Related Art

When describing relevant art, it is important to consider that the present invention is capable of securing samples of a variety of different substances. These substances include chemicals in liquid or gas form, and thus Solid Phase Microextraction (SPME) is one field in which the present invention finds application. However, while SPME is typically associated with the extraction of chemicals from liquids and vapors, the present invention can also obtain samples from solids and suspensions of solids.

SPME is known to those skilled in the art as a technique for sampling and concentrating chemical compounds for analysis by chromatography or other methods. Typically, a fiber is used to extract analytes from a sample and deliver them for analysis. The fiber is typically made of a fused silica or metal fiber coated with a polymer or an absorbent that is used to capture and concentrate the analytes by partition or adsorption. The fiber is moved to a sample introduction port of a chromatograph or spectrometer for desorption or extraction for analysis. The fiber used for SPME is typically held in a syringe-like device for convenience. The fiber is easily protected and transferred within the walls of a protective sheath that extends outwardly from the syringe-like device.

Before proceeding with the description of SPME techniques, it is noted that the present invention is using a syringe-like device for sampling, concentration, transporting and injecting samples. While the syringe-like device does not store a liquid within its housing like an actual syringe, the housing of the present invention is constructed to appear like a syringe with a handle, plunger, and a needle-like protrusion that is actually a sheath for the fiber described above. Accordingly, the term “syringe” used in this document and the claims is the syringe-like device to be more fully described hereinafter, and should not be mistaken for an actual syringe.

One of the drawbacks of existing syringes used for SPME is that two hands are typically needed to operate it. For example, a first hand grips the shaft of the syringe while the second hand extracts a plunger to move the fiber into the syringe after a sample has been taken. Thus, the process of obtaining a sample requires two hands. Accordingly, it would be an advantage over the state of the art in SPME syringes to provide a syringe that can be operated with only one hand, leaving the second hand free for other tasks.

Understandably, SPME is not the only application of the present invention that must be considered. It is clearly another aspect of the present invention to be able to use the syringe to also obtain samples from solids and solids in suspensions. For example, solids can be collected on filaments, as opposed to fibers used to collect samples from liquids and gases. The filaments of the present invention are designed with cavities, apertures or other similar features that enable solids to be collected on or within the filament. The present invention also provides a means for collecting solids in suspensions, as will be disclosed.

Samples also need to be carefully tracked from a point of origin, through transport, to final analysis. It would be another advantage over the state of the art to provide the syringe described above having a fiber or filament, and also including means for electronically tracking a chain of custody of samples.

### BRIEF SUMMARY OF THE INVENTION

The present invention is a syringe-like device (hereinafter “syringe”) that is operable by a single hand, wherein the syringe includes a plunger for ejecting and then retracting a fiber or filament used for the collection of solids, solids in suspensions and liquids, wherein the syringe includes a microchip embedded in the syringe housing, and wherein the microchip enables electronic chain of custody tracking of a sample from a point of origin through final analysis.

These and other objects, features, advantages and alternative aspects of the present invention will become apparent to those skilled in the art from a consideration of the following detailed description taken in combination with the accompanying drawings.

### BRIEF DESCRIPTION OF THE SEVERAL VIEWS OF THE DRAWINGS

FIG. 1 is a solid perspective view of an assembled syringe as taught in accordance with the principles of the present invention.

FIG. 2 is a wireframe perspective view of an assembled syringe as taught in accordance with the principles of the present invention.

FIG. 3 is an exploded wireframe perspective view of the components used in the syringe as shown in FIGS. 1 and 2.

FIG. 4 is provided as an illustration of a receiving port on a sample analysis device that is designed for receiving a sample from the syringe of the present invention.

FIG. 5 is provided as an example of ID circuit placement within a portion of the handle of the syringe.

FIG. 6 is an electrical circuit diagram of a circuit that provides access to memory of an ID circuit disposed in the body of a syringe.

FIG. 7 is an electrical circuit diagram of a circuit of a recording instrument that provides access to the memory of an ID circuit.

FIG. 8 is an illustration of the evaporation process of solids in suspension on a twisted filament.

FIG. 9 is an illustration of three wires that form a whisk for obtaining samples of solids and solids in suspensions.

### DETAILED DESCRIPTION OF THE INVENTION

Reference will now be made to the drawings in which the various elements of the present invention will be given numerical designations and in which the invention will be discussed so as to enable one skilled in the art to make and use the invention. It is to be understood that the following description is only exemplary of the principles of the present invention, and should not be viewed as narrowing the claims which follow.

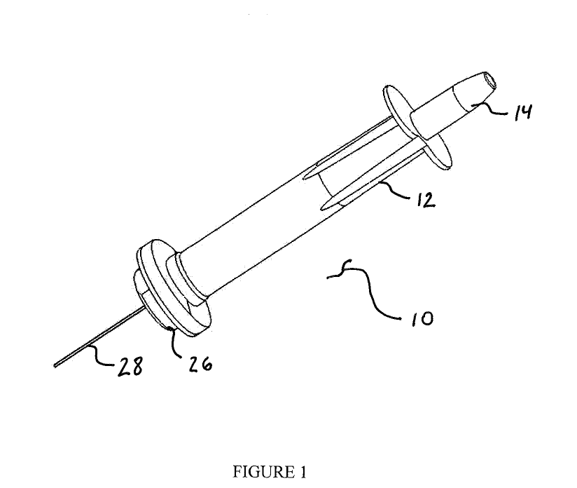

FIG. 1 is provided as a solid perspective view of a first embodiment of the present invention for a syringe 10 that can be operated using only one hand. The components that are visible in this figure include a housing or handle 12, a thumb actuator 14, a ground cap 26, and an outer housing or sheath 28 for the filament or fiber (hereinafter to be referred to collectively as a “fiber”).

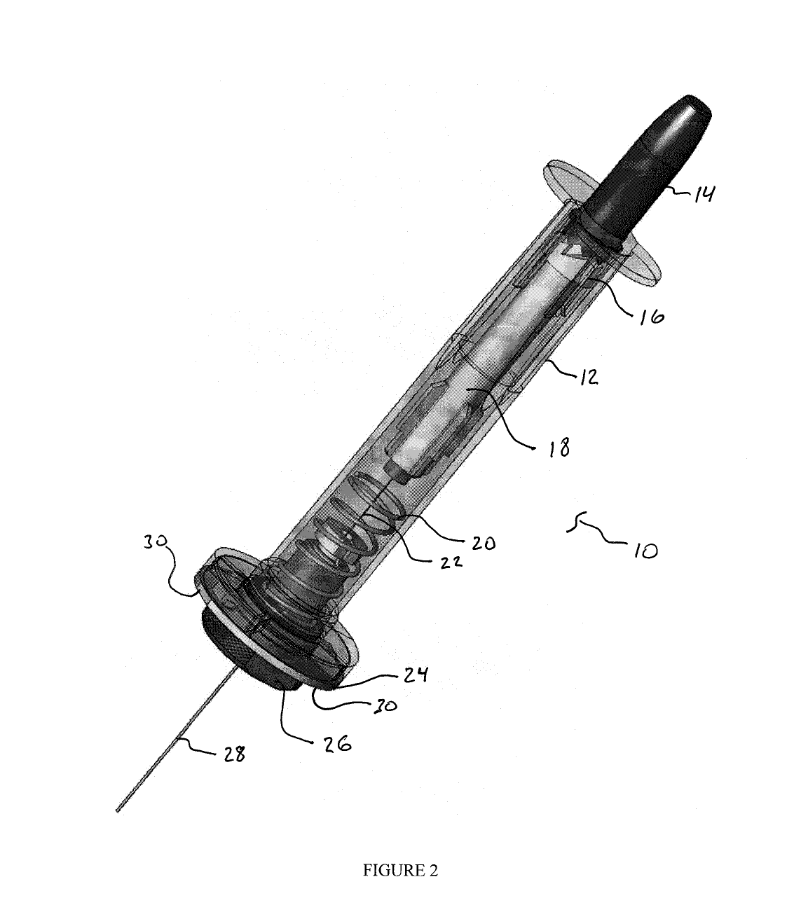

FIG. 2 is provided as a wire-frame perspective view of the first embodiment of the present invention as shown in FIG. 1. The components that are visible in this figure include the housing or handle 12, the thumb actuator 14, a cam mechanism 16, a plunger 18, a spring 20, a filament or fiber 22 suitable for obtaining samples to be analyzed from liquids, vapors, solids or solids in suspensions, an identification (ID) circuit 24, the ground cap 26, the outer housing or sheath 28 for the fiber, and electrodes 30 disposed on an underside of the ID circuit 24.

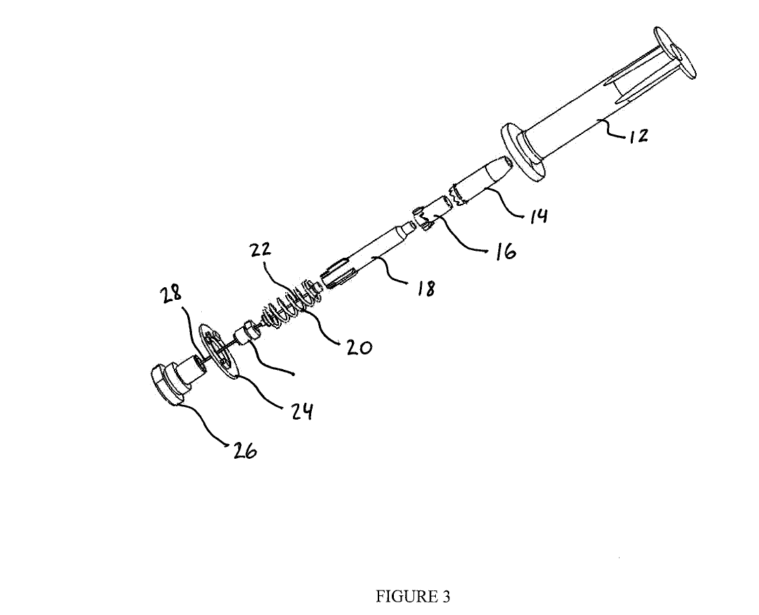

FIG. 3 is provided to show all of the components in an exploded view of the syringe 10 from FIG. 1, wherein the syringe is capable of single-handed operation when extracting, transporting, and delivering a sample for analysis. As before, the components include the housing or handle 12, the thumb actuator 14, the cam mechanism 16, the plunger 18, the spring 20, the fiber 22, the identification (ID) circuit 24, the ground cap 26, and the outer housing or protective sheath 28 for the fiber. A fiber clamp 32 is provided to secure the fiber within the handle 12. It is noted that two other components are not visible in this view, but are nonetheless included in the components shown. These components are two seals disposed within the ground cap 26.

Operation of the syringe 10 of the first embodiment using only one hand is relatively straightforward. In a first embodiment, a user grasps the handle 12 with all of the fingers of a hand, leaving the thumb disposed over the thumb actuator 14. Alternatively, the user can grasp the handle 12 between an index finger and a middle finger of one hand. The thumb is then placed on top of the thumb actuator 14 such that it is ready for operation in either embodiment.

The user is free to perform other tasks with the hand that is not holding the syringe 10. These tasks include such things as securing a sample container while the sample is being obtained, preparing analysis equipment for sample introduction, and holding another device. Thus, it is immediately apparent that the user is free to do any other task with the free hand, and will typically be free to perform a task that would otherwise need to be performed by another person. Thus, not only does the present invention enable a user to perform more than one task while taking a sample, but also enables samples to be taken in situations where only a single person is present to perform the task.

It is noted for clarity that hereinafter, the term “sample” refers to liquids, solids, and solids in suspensions of any substance that can be sampled using the fiber of the present invention.

A more detailed description of the operation of the syringe 10 is herein described for the first embodiment. The syringe 10 of the present invention includes a camming system in order to lock the fiber 22 into an extended position when actuated a first time, and unlock and retract the fiber when actuated a second time. The spring 20 is required to make the camming system function as indicated above.

An important insight into the invention is that the spring 20 that is used in this first embodiment may be replaced by any suitable spring-like device that provides the needed tension for the cam mechanism 16 to function. Similarly, as the spring 20 and cam mechanism 16 function as a system to enable the fiber 22 to be ejected from and retracted into the protective sheath 28, it is an aspect of the present invention that this system can be replaced by any equivalent means for providing this function of fiber ejection and retraction.

Continuing with the first embodiment, when the syringe 10 is ready for use, the fiber 22 is loaded into the handle 12 and secured via the fiber clamp 32. For example, the fiber clamp 32 can be a small nut. The fiber clamp 32, along with the seals, prevents any chemicals from moving into the handle 12. The fiber clamp 32 also makes it possible to remove and replace the fiber 22 after it has been used to secure a sample. However, it is envisioned that the syringe 10 will be inexpensive enough such that it can be a disposable item. Nevertheless, it is envisioned that the fiber 22 may be replaced and the syringe 10 reused if desired.

When the user is ready to take a sample using the syringe 10, the user must lock the fiber 22 into an ejected position so that the sample can be disposed on the fiber through absorption, adsorption, static charge, etc. The user uses a digit (typically a thumb) on the thumb actuator 14. The thumb actuator 14 slides into the handle 12 until it reaches a point where the camming system prevents the fiber 22 from being retracted into the protective sheath 28. At this time in the procedure, the user does not have to keep a digit on the thumb actuator 14 in order to keep the fiber 22 extended from the protective sheath 28. The user then holds the fiber 22 in the sample for an appropriate amount of time as known to those skilled in the art.

In this first embodiment of the syringe 10, the user presses the thumb actuator 14 again in order to move the cam mechanism 16 to a different position that allows the fiber 22 to be retracted within its protective sheath 28. The syringe 10 is then moved to storage or taken to a chemical analysis station where the sample absorbed by the fiber 22 is retrieved and analyzed.

It should be noted that the camming system as described in the first embodiment can be modified to operate in a different manner. For example, a camming mechanism can also be actuated by the thumb, but include a locking release that is reachable on an outside of the handle 12. Thus, releasing the extended fiber 22 would consist of moving a tab or other release means that is disposed on the side of the handle 12.

It is noted that once the fiber 22 has been extended from outside its protective sheath 28, it is also possible to attach the syringe 10 to a container holding a sample. Thus, the syringe 10 enables “hands-free” operation while the sample is being absorbed. However, this is an optional aspect of the present invention, and not a requirement of the first embodiment.

Once the sample has been obtained, the fiber 22 is retracted inside the protective sheath 28. The protective sheath 28 not only protects the sample that has been absorbed by the fiber 22, but also protects the user or other persons present from the chemical in the sample. The user releases the cam mechanism 16 and retracts the fiber 22 inside the protective sheath 28 by actuating the thumb actuator 14 a second time.

The first embodiment described above describes a camming system that locks the fiber 22 into a single desired position external of its protective sheath 28 while the sample is being obtained. However, in an alternative embodiment, it is envisioned that the camming system incorporates means for enabling the fiber 22 to be exposed in ever-increasing lengths outside the protective sheath 28. Accordingly, it is envisioned that in one alternative embodiment, the fiber 22 may be exposed using pre-set incremental lengths upon multiple actuations of the thumb actuator 14 until the fiber is exposed to a maximum ejected length.

In yet another alternative embodiment, it is envisioned that the fiber 22 may be exposed in a non-incremental manner, and to any desired non-predetermined length, up to the maximum length of the fiber. It is envisioned that the fiber 22 would thus be ejected a length that is directly proportional to a length that the thumb actuator 14 was pushed into the handle 12.

Once the sample has been obtained, the sample is now safely moved to, for example, an analysis device or a storage device. Examples of the types of analysis that can be performed include mass spectrometry, ion mobility spectrometry, gas chromatography, liquid chromatography, flow injection analysis, etc. What is important to the present invention is that the analysis devices include an injection port for receiving the syringe 10.

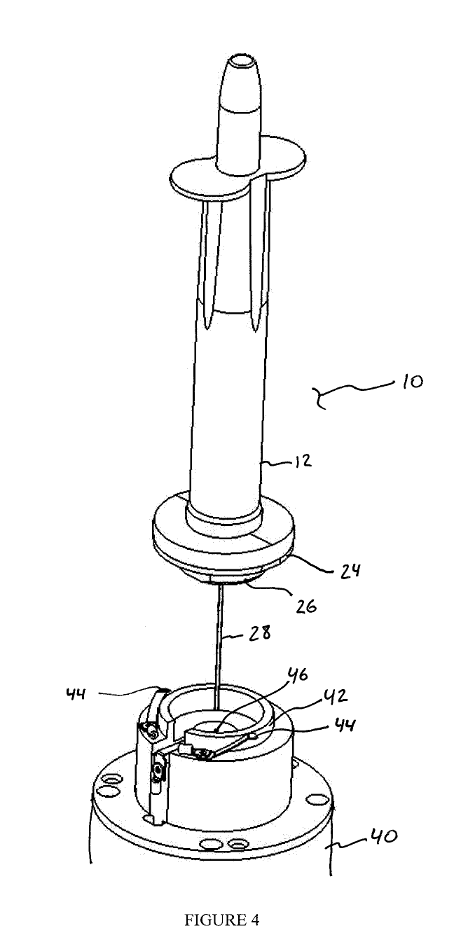

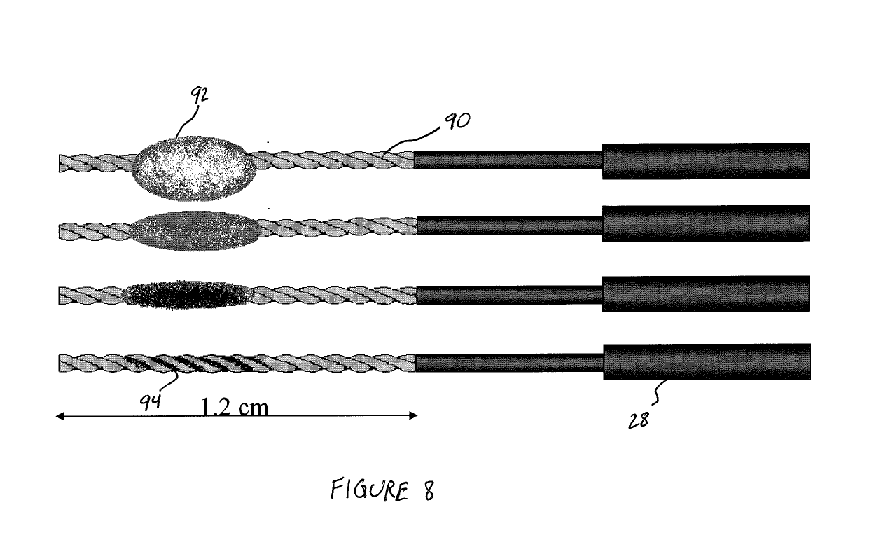

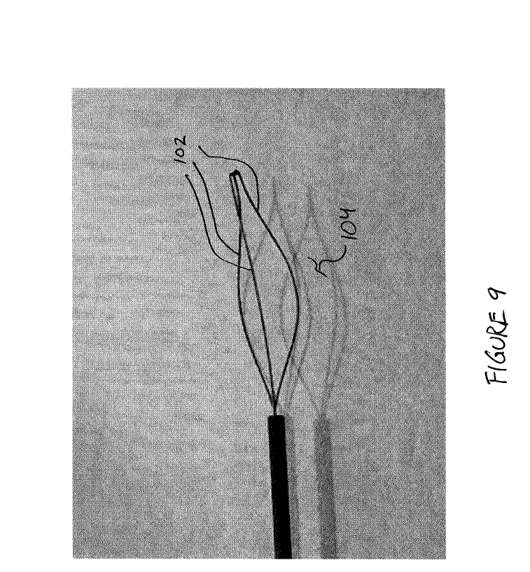

FIG. 4 is provided as an illustration of an analysis device 40 that is designed for receiving the syringe 10. The analysis device itself is not an element of the present invention. However, the injection port 42 shown in FIG. 4 is relevant in that it includes a circuit, shown in FIG. 7, for communicating with the ID circuit 24. The receiving port 42 can be any desired shape so long as two criteria are met. First, the injection port 42 must have an aperture 46 for receiving the protective sheath 28. Second, the injection port 42 must provide a surface whereon at least two electrodes 44 can be disposed, wherein the at least two electrodes must be capable of making electrical contact with the two electrodes 30 of the syringe 10. Beyond those two requirements, the analysis device 40 is limited only by its own needs.

Alternatively, the syringe 10 can be coupled to the receiving port 42 by twisting the handle 12, thereby mechanically locking the syringe to the receiving port by providing complementary locking channels in the receiving port. This alternative embodiment would enable hands-free delivery of the sample once the fiber has been ejected into the analysis device 40.

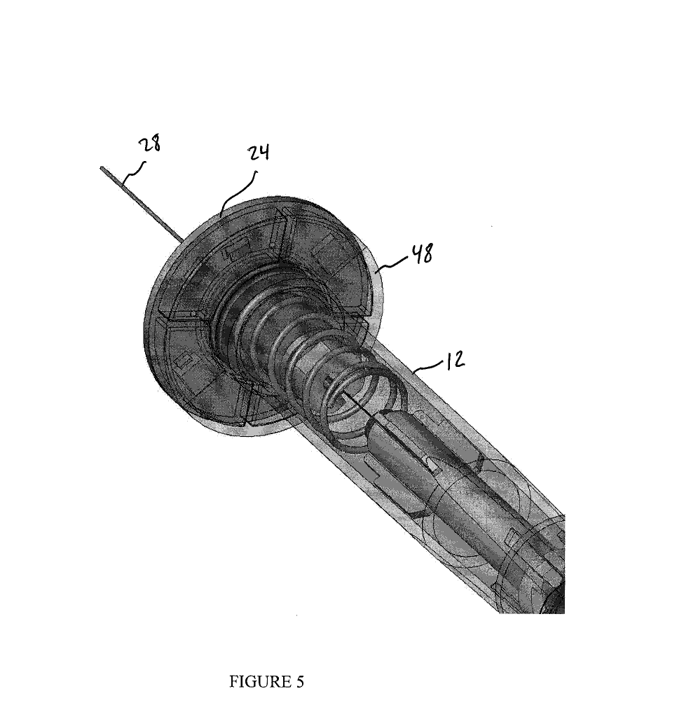

Another important aspect of this first embodiment of the present invention is the embedding of a microchip with memory into the syringe 10. The microchip is used to uniquely identify the sample absorbed by the fiber 22. Thus, the microchip and memory will be referred to collectively hereinafter as an identification (ID) circuit 24. Exact placement of the ID circuit 24 in this first embodiment is near the fiber ejection and retraction end thereof so that it can be easily placed in electrical contact with a device capable of reading data from or writing data to the ID circuit. FIG. 5 is provided as an example of one possible location where the ID circuit 24 can be disposed within part of the handle 12 of the syringe 10. More specifically, a flange 48 on the handle 12 can have disposed underneath it the ID circuit 24 disposed on its own circuit board. However, it should be noted that the placement of the ID circuit 24 may be altered without materially affecting operation of the present invention. It is only important that the ID circuit 24 be positioned such that it can communicate with electrical contacts within the receiving port 42.

The ID circuit 24 enables tracking of a sample disposed on the fiber 22 from a point of origin (where the sample was obtained), through a chain of custody, to final analysis and/or storage. The electronic chain of custody is maintained by employing a recording instrument that can read from and add information to the data stored in the ID circuit 24. The recording instrument can be a stand-alone device having its own interface, or it can be coupled to another device such as a computer that provides an interface. This means that the recording instrument can operate in a stand-alone mode of operation, or be dependent upon another device for communication. The mode of operation is thus independent of any connection to or separation from a chemical analyzer such as the analysis device 40 shown in FIG. 4.

In this first embodiment, the ID circuit 24 is any digital memory module and I/O circuitry that enables storage and reading of data that can maintain the electronic chain of custody of the sample. The memory is preferably non-volatile so that data remains safely within the memory even after power is removed. The use of non-volatile memory eliminates any need for a battery in the syringe 10 to preserve data in the memory.

In an alternative embodiment of the present invention, it is envisioned that the ID circuit 24 is simplified even further, and consists only of a memory module. Thus, any recording instrument would have to provide the means for communicating with the memory.

When considering the nature of the memory being used in the first embodiment of the present invention, it is noted that data stored therein cannot be erased after being stored. Thus, while new information can be added to the memory, old information is always retained. The memory can also be read as often as desired without modifying data.

In an alternative embodiment, it is envisioned that a reusable syringe includes an ID circuit 24 that uses memory that can be erased completely. It is important that the memory erasure cannot be selective, to thereby avoid doubt as to the integrity of the electronic chain of custody.

In another alternative embodiment, it is envisioned that a reusable syringe has a replaceable fiber for collecting a sample, and a new ID circuit 24. The fiber 22 and ID circuit 24 would be a single unit that would again ensure integrity of the electronic chain of custody.

Another important aspect of the memory of the first embodiment is that it has stored therein a unique code for the ID circuit 24. By giving each ID circuit 24 a unique code, electronic chain of custody is again ensured because there will be no possibility that two ID circuits will have the same code. Thus, even if the syringe is reusable, a new unique code would still need to be provided for the memory used in the ID circuit 24.

Storing data in the memory of the ID circuit 24 can be accomplished using an appropriate physical and electrical connection. Systems and methods for storing data to and reading data from non-volatile memory are well known to those skilled in the art, and thus the means for accomplishing these tasks is not of particular importance to the present invention. It is the application of memory storage and retrieval techniques as applied to the task of electronic chain of custody that is important for the present invention.

One reason for the requirement of the first embodiment that a physical connection be made between the syringe 10 and the recording instrument before data can be stored on the ID circuit 24, is that this step prevents unwanted tampering with the data stored therein. Thus, it is inappropriate to provide wireless means, such as radio-frequency (RF) or infra-red (IR) means, for storing data in the memory of the ID circuit 24. However, it is possible that RF or IR means for reading data may be permissible, and should be considered to be within the scope of an alternative embodiment of the present invention.

The data that is stored in the non-volatile memory of the ID circuit 24 to create the electronic chain of custody can be selected from information that is typically considered useful for such purposes. For example, such information may include, but should not be considered limited to, time and date that the sample is being taken, the location where the sample is being obtained, an identification code for the operator performing the sampling, storage, or analysis, and a unique identification code for the recording instrument.

The recording instrument that is used to store data to and read data from the ID circuit 24 can be a portable or stationary device. Thus, the recording instrument can be battery powered, or be operated directly from current from a wall socket.

Access to the recording instrument can be provided by any convenient interface. For example, a computer can provide access to the recording instrument and from there to the ID circuit 24 via a USB interface, Firewire, or any other wired access protocol or hardware connection. As mentioned previously, wireless access means might also be provided to read the data stored in the ID circuit 24 in an alternative embodiment. In this case, the ID circuit 24 must also include means for transmitting data stored therein.

The recording instrument might also include a display and keyboard so that it does not have to be accessed through a computer, thereby making it a true stand-alone device. However, size and complexity of the recording instrument will most likely be reduced by providing access through a computer, and thereby avoiding the need for keyboard and display on the recording instrument itself.

A specialized version of the recording instrument is designated as an Analyzer Recording Instrument. The Analyzer Recording Instrument may be designed to provide an interface with a particular analyzer. In the first embodiment, the Analyzer Recording Instrument is configured so that it is pre-programmed with all of the specific requirements of the particular analyzer with which it operates.

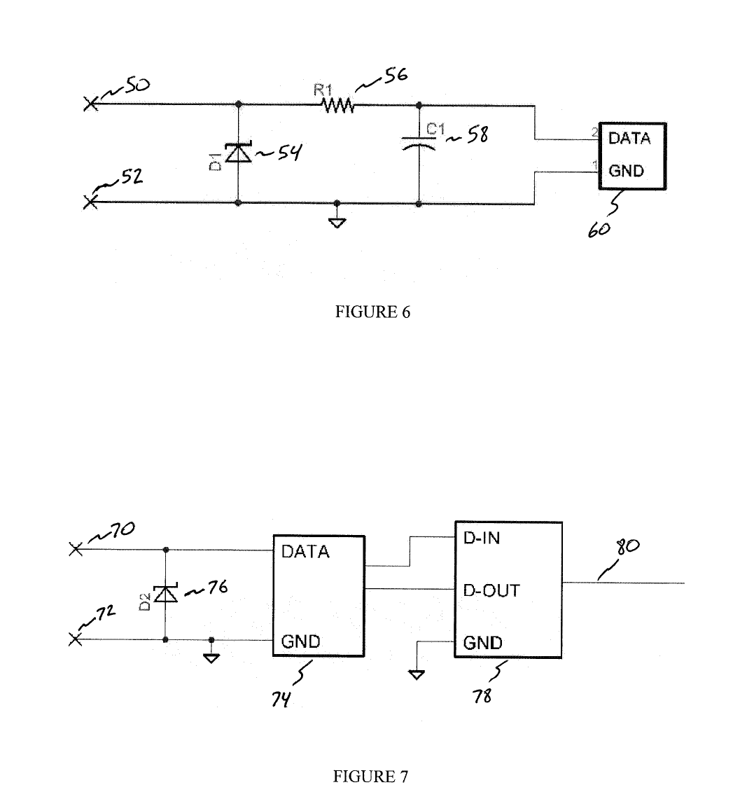

FIG. 6 is provided as an electrical circuit diagram of one possible embodiment of an electrical circuit that can be used for accessing the ID circuit 24 on the syringe 10. The electrical circuit shows a first electrical connection 50 and a second electrical connection 52, a diode 54, a resistor 56, a capacitor 58, and a non-volatile memory device 60.

To complement the circuit of FIG. 6, FIG. 7 is provided as an electrical circuit diagram of one possible embodiment of an electrical circuit that can be used in a recording instrument that the ID circuit 24 is coupled to in order to gain access and send data or retrieve data. FIG. 7 shows a first electrical connection 70, a second electrical connection 72, an I/O driver chip 74, a diode 76, and a microcontroller 78. The microcontroller 78 will include access via line 80 to either an external I/O port for communicating with an external computer, or access to an internal CPU if the recording instrument is acting as a stand-alone device.

While the ID circuit 24 has application for providing chain of custody for a sample absorbed by a fiber 22, the ID circuit can also be used in other devices. For example, a typical hypodermic syringe, a filter for collection of samples from air or other gaseous environments, and a plug sample device for securing samples of solid materials.

Several aspects of the present invention that bear further explanation begin with the fiber 22 when it is formed as a filament to collect liquids, solids or solids in suspensions. Collecting solids in suspensions is illustrated in FIG. 8. FIG. 8 shows a twisted wire 90 having a drop 92 of a solid in suspension disposed thereon. The liquid if the suspension is evaporated away as show in the four illustrations of the twisted wire 90 and the gradually disappearing liquid of the suspension, until all that remains on the twisted wire is the solid 94 that was in the suspension. The solid has precipitated between and around the wires of the twisted wire 90. The twisted wire 90 (which is fiber 22) can now be retracted into the protective sheath 28.

A twisted wire 90 should not be considered the only alternative embodiment for collecting samples. For example, braided wires, or a wire with holes drilled therethrough can also provide the desired cavities where solids can be disposed.

Another system for the collection of liquids, solids and solids in suspensions is illustrated by the use of a whisk 100 as shown in FIG. 9. FIG. 9 shows three wires 102 that are arranged in a whisk 104 shape. Any suitable whisk-like shape that accomplishes the function to be described may be substituted for the design shown in FIG. 9. What is important is that solids in suspensions can be obtained by disposing the whisks 104 in the liquid, withdrawing the whisk, and evaporating the liquid in the sample just as was done for the liquid in FIG. 8.

As a last aspect of the invention, a suitable whisk 104 can be created using the following procedure. Three nitinol wires are welded together at one end. The three loose ends of the wires are inserted into a stainless steel tube having an inner diameter of 0.006 inches. A metal spacer is then inserted between the three wires to separate the wires and form the whisk 104. The wires and tube are then disposed in an oven at 500° C. for 5 minutes. The spacer can now be removed, and the memory effect induced on the three wires will now maintain the whisk 104 in the desired shape. The three wires are now removed from the tube, and coupled to a holder that is inserted into the syringe 10.

It is to be understood that the above-described arrangements are only illustrative of the application of the principles of the present invention. Numerous modifications and alternative arrangements may be devised by those skilled in the art without departing from the spirit and scope of the present invention. The appended claims are intended to cover such modifications and arrangements.

What is claimed is:

1. A syringe suitable for obtaining a sample of a substance to be analyzed, wherein the syringe only requires a single hand to operate, and said syringe comprised of:
a fiber suitable for collecting a sample from a substance to be analyzed;
a protective sheath for protecting the fiber, wherein the fiber can be extracted from the protective sheath and retracted back inside;
a plunger for actuating a means for ejecting and retracting the fiber from the protective sheath; and
a thumb actuator coupled to the plunger to thereby push the plunger when fingers grasp the handle and push on the thumb actuator to thereby operate the ejecting and retracting means using a single hand.

2. The syringe as defined in claim 1 wherein the means for ejecting and retracting the fiber is further comprised of:
a cam mechanism disposed inside a handle for locking the fiber into an ejected position; and for releasing the fiber so that it can be retracted; and
tension means for providing tension for the cam mechanism.

3. The syringe as defined in claim 2 wherein the cam mechanism further comprises a locking position wherein the cam mechanism is held under tension by the tension means when the thumb actuator is pushed a first time to eject the fiber from the protective sheath, and wherein the thumb actuator can be released while the fiber remains in the ejected position.

4. The syringe as defined in claim 3 wherein the cam mechanism further comprises an unlocking position wherein the cam mechanism is released from tension when the thumb actuator is pushed a second time, thereby enabling the fiber to be retracted within the protective sheath.

5. The syringe as defined in claim 3 wherein the cam mechanism further comprises an unlocking position wherein the cam mechanism is released from tension when a release means on a side of the handle is actuated, thereby enabling the fiber to be retracted within the protective sheath.

6. The syringe as defined in claim 3 wherein the cam mechanism further comprises means for enabling the fiber to be ejected in an incremental manner in response to multiple actuations of the thumb actuator until reaching a maximum ejected length of the fiber.

7. The syringe as defined in claim 3 wherein the cam mechanism further comprises means for enabling the fiber to be ejected to a non-incremental length that is directly proportional to a length that the thumb actuator is pushed within the handle.

8. The syringe as defined in claim 1 wherein the syringe further comprises an Identification (ID) circuit disposed thereon, wherein the ID circuit enables electronic chain of custody of a sample obtained by the fiber.

9. The syringe as defined in claim 8 wherein the syringe further comprises at least two electrical connections on an outer surface thereof for enabling communication with the ID circuit.

10. The syringe as defined in claim 9 wherein the ID circuit further comprises at least one memory module, wherein the memory module is non-volatile to thereby enable data retention after power is removed from the ID circuit.

11. The syringe as defined in claim 10 wherein the at least one memory module further comprises memory that can only be written to and read from, but not erased, to thereby ensure the integrity of the electronic chain of custody of the sample.

12. The syringe as defined in claim 10 wherein the at least one memory module further comprises memory that can be written to, read from, and erased in totality, to thereby ensure that if the at least one memory module is being reused, the integrity of the electronic chain of custody of the sample is ensured.

13. The system as defined in claim 1 wherein the system further comprises a recording instrument, wherein the recording instrument can write data to and read data from the ID circuit.

14. The system as defined in claim 13 wherein the recording instrument is a stand-alone device having its own interface to a user such that information can be written to and read from the ID circuit in accordance with commands entered into the recording instrument.

15. The system as defined in claim 13 wherein the recording instrument is coupled to a second device, wherein the second device provides an interface such that information can be written to and read from the ID circuit in accordance with commands entered into the second device.

16. The system as defined in claim 13 wherein the recording instrument is capable of being powered by a battery or an external power source.

17. The system as defined in claim 13 wherein the system further comprises mechanical means for mechanically coupling the syringe to the recording instrument to thereby enable the transfer of the sample to the recording instrument in a hands-free procedure.

18. The syringe as defined in claim 1 wherein the syringe further comprises wireless communication means, wherein the wireless communication means can only be used to transmit data from the ID circuit.

19. The system of a syringe and recording instrument as defined in claim 13 wherein the recording instrument is further comprised of an Analyzer Recording Instrument, wherein the Analyzer Recording Instrument is coupled to an analyzer, and wherein the Analyzer Recording Instrument is specifically designed to operate with a specific analyzer to thereby enhance operation of the system.

20. The system of a syringe and recording instrument as defined in claim 13 wherein the recording instrument is further comprised of:
a first electrical contact coupled to a data input of an I/O driver and an output of a first diode,
a second electrical contact coupled to a ground input of the I/O driver and an input of the first diode; and
a microcontroller coupled to the I/O driver at a Data-In connector and a Data-out connector, and wherein the microcontroller also includes means for communicating to an Internal CPU or an external communications port.

21. The syringe as defined in claim 2 wherein the tension means for providing tension for the cam mechanism is further comprised of a spring.

22. A method for obtaining a sample of a substance to be analyzed by using a syringe that only requires one hand to operate and thereby obtain the sample, said method comprising the steps of:
(1) providing a syringe having a housing wherein is disposed a fiber, a protective sheath, a plunger for actuating means for means for ejecting and retracting the fiber from the protective sheath, and a thumb actuator coupled to the plunger;
(2) grasping the housing of the syringe using fingers of a first hand;
(3) placing a thumb of the first hand on the thumb actuator; and
(4) pushing the thumb actuator with the thumb a first time to thereby actuate the cam mechanism using only the first hand.

23. The method as defined in claim 22 wherein the method further comprises the steps of:
(1) using a cam mechanism disposed inside a handle for locking the fiber into an ejected position; and for releasing the fiber so that it can be retracted; and
(2) using a spring to provide tension for the cam mechanism.

24. The method as defined in claim 23 wherein the method further comprises the step of pushing the thumb actuator with the thumb a second time to thereby release the cam mechanism and cause the fiber to be retracted within the protective sheath.

25. The method as defined in claim 23 wherein the method further comprises the steps of:
(1) providing a first locking position for the cam mechanism; and
(2) holding the cam mechanism under tension using the spring when the thumb actuator is pushed the first time to eject the fiber from the protective sheath; and
(3) keeping the fiber in the ejected position while releasing the thumb actuator.

26. The method as defined in claim 23 wherein the method further comprises the steps of:
(1) providing an unlocking position for the cam mechanism; and
(2) releasing the tension of the spring by moving the cam mechanism to the unlocking position when the thumb actuator is pushed the second time; and
(3) retracting the fiber within the protective sheath.

27. The method as defined in claim 26 wherein the method further comprises the steps of:
(1) providing means for incrementally ejecting the fiber in response to multiple actuations of the thumb actuator; and
(2) actuating the thumb actuator a plurality of times until reaching a maximum ejected length of the fiber.

28. The method as defined in claim 26 wherein the method further comprises the steps of:
(1) providing means for non-incrementally ejecting the fiber; and
(2) actuating the thumb actuator to thereby eject the fiber a length that is directly proportional to a length that the thumb actuator is pushed within the housing.

29. The method as defined in claim 22 wherein the method further comprises the steps of:
(1) providing an Identification (ID) circuit in the syringe; and
(2) recording information on the ID circuit to thereby enable tracking of an electronic chain of custody of a sample obtained by the fiber.

30. The method as defined in claim 29 wherein the method further comprises the steps of:
(1) providing at least two electrical connections to the ID circuit on an outer surface of the housing; and
(2) storing information on the ID circuit via the at least two electrical connections.

31. The method as defined in claim 30 wherein the method further comprises the step of providing at least one non-volatile memory module in the ID circuit, wherein the memory module retains information stored therein after power is removed from the ID circuit.

32. The method as defined in claim 31 wherein the method further comprises the steps of:
(1) enabling reading from the at least one non-volatile memory module an unlimited number of times;
(2) enabling writing to the at least one non-volatile memory module until memory capacity is reached; and
(3) preventing erasure of any data from the at least one non-volatile memory module to thereby ensure the integrity of the electronic chain of custody of the sample.

33. The method as defined in claim 32 wherein the method further comprises the steps of:
(1) enabling reading from the at least one non-volatile memory module an unlimited number of times;
(2) enabling writing to the at least one non-volatile memory module until memory capacity is reached; and
(3) enabling complete erasure of all data from the at least one non-volatile memory module if the memory module is to be reused, to thereby ensure the integrity of the electronic chain of custody of the sample.

34. The method as defined in claim 22 wherein the method further comprises the step of providing a recording instrument writing to and reading data from the ID circuit.

35. The method as defined in claim 34 wherein the method further comprises the step of providing a stand-alone recording instrument, wherein the recording instrument provides its own interface to a user such that information can be written to and read from the ID circuit in accordance with commands entered into the recording instrument.

36. The method as defined in claim 34 wherein the method further comprises the steps of:
(1) coupling the recording instrument to a second device; and
(2) operating the second device as an interface to the recording instrument, wherein information can be written to and read from the ID circuit in accordance with commands entered into the second device.

37. The method as defined in claim 36 wherein the method further comprises the steps of:
(1) powering the recording instrument with a battery when the recording instrument is operated away from an external power source; and
(2) powering the recording instrument with an external power source when the external power source is available.

38. The method as defined in claim 36 wherein the method further comprises the step of providing a mechanical means for mechanically coupling the syringe to the recording instrument to thereby enable the transfer of the sample to the recording instrument.

39. The method as defined in claim 29 wherein the method further comprises the steps of:
(1) providing wireless communication means; and
(2) only transmitting data from the circuit ID.

40. The method as defined in claim 34 wherein the method further comprises the steps of:
(1) providing an Analyzer Recording Instrument; and
(2) coupling the Analyzer Recording Instrument to a chemical analyzer, wherein the Analyzer Recording Instrument is specifically designed to operate with a specific chemical analyzer to thereby enhance operation of the system.

41. A syringe suitable for obtaining a sample of a substance to be analyzed, wherein the syringe only requires a single hand to operate, and said syringe comprised of:
a fiber suitable for collecting a sample from a substance to be analyzed;
a protective sheath for protecting the fiber, wherein the fiber can be extracted from the protective sheath and retracted back inside;
a plunger for actuating a means for ejecting and retracting the fiber from the protective sheath;
a thumb actuator coupled to the plunger to thereby push the plunger when fingers grasp the handle and push on the thumb actuator to thereby operate the ejecting and retracting means using a single hand; and
an Identification (ID) circuit disposed thereon, wherein the ID circuit enables electronic chain of custody of a sample obtained by the fiber.

42. The syringe as defined in claim 41 wherein the fiber is selected from the group of fibers comprised of SPME fibers, twisted wires, braided wires, and wires forming a whisk.

43. A syringe suitable for obtaining a sample of a substance to be analyzed, wherein the syringe only requires a single hand to operate, and said syringe comprised of:
a fiber suitable for collecting a sample from a substance to be analyzed;
a protective sheath for protecting the fiber, wherein the fiber can be extracted from the protective sheath and retracted back inside;
a plunger for ejecting the fiber from the protective sheath;
a cam mechanism disposed inside a handle for locking the fiber into an ejected position; and for releasing the fiber so that it can be retracted;
a spring inside the handle for providing tension for the cam mechanism; and
a thumb actuator coupled to the plunger to thereby push the plunger when fingers grasp the handle and push on the thumb actuator to thereby operate the cam mechanism using a single hand.
